(19) United States
(12) Patent Application Publication
(10) Pub. No.: US 2023/0321460 A1
(43) Pub. Date: Oct. 12, 2023

(54) SLAT FOR COLLIMATING THERAPY RADIATION

(71) Applicant: Siemens Healthcare GmbH, Erlangen (DE)

(72) Inventors: Olivia Noack, Bayreuth (DE); Sebastian Graebner, Speichersdorf (DE); Christian Helldoerfer, Kirchenthumbach (DE); Theresa Haeupl, Ahorntal (DE); Georg Walberer, Kastl (DE); Franz Dirauf, Bad Staffelstein (DE); Michael Langguth, Wipfratal (DE)

(73) Assignee: Siemens Healthcare GmbH, Erlangen (DE)

(21) Appl. No.: 18/296,066

(22) Filed: Apr. 5, 2023

(30) Foreign Application Priority Data
Apr. 8, 2022 (DE) 10 2022 203 544.5

(51) Int. Cl. A61N 5/10

(52) U.S. Cl. CPC A61N 5/1045; A61N 2005/1092

(57) ABSTRACT

The invention relates to a slat for collimating therapy radiation, comprising a collimation region made from a first material; and a holding region made from a second material, wherein the collimation region and the holding region are connected together by a connection point, the first material is configured to collimate therapy radiation, and the holding region is couplable to an adjusting facility for adjusting the slat.

### CROSS-REFERENCE TO RELATED APPLICATION(S)

The present application claims priority under 35 U.S.C. § 119 to German Patent Application No. 10 2022 203 544.5, filed Apr. 8, 2022, the entire contents of which are incorporated herein by reference.

### FIELD

One or more example embodiments of the present invention relates to a slat for collimating therapy radiation. One or more example embodiments of the present invention also relates to a collimator and to a method for producing a slat for collimating therapy radiation.

### RELATED ART

It is known to carry out radiation therapy, for example for treating a tumor or also for treating a benign disease, such as heel spurs, tennis elbow, shoulder pain, osteoarthritis of the various joints and vertebral hemangioma. In this context, therapy radiation is emitted onto a treatment area of an examination object, for example the tumor or the affected limbs. The therapy radiation can be, in particular, high-energy electromagnetic radiation generated with a linear accelerator, in particular X-ray radiation. Alternatively, therapy radiation can be particle radiation, in particular proton radiation or heavy ion radiation or alpha radiation, etc.

A region that can be irradiated is delimited by a radiation field of the therapy radiation. In order to protect surrounding tissue and/or organs of the examination object inside the radiation field but outside of the treatment area from the therapy radiation, the therapy radiation is collimated during radiation therapy. For this, a plurality of slats is typically arranged or oriented in the radiation field between a source of the therapy radiation and the examination object in such a way that only the treatment area to be irradiated is not covered by a slat in the radiation field. One slat of the plurality of slats is embodied to strongly attenuate or absorb the therapy radiation in such a way that exposure to radiation or an intensity of the therapy radiation behind the slat is negligibly low. “Behind” describes the arrangement from the perspective of the source of the therapy radiation. In particular, the examination object is arranged “behind” the slat. In particular, the region of the slat, which is positioned in the radiation field of the therapy radiation, hereinafter referred to as the collimation region, thus has to be made from a material that attenuates the therapy radiation. For this, the slat is typically composed of tungsten or a compound comprising tungsten or a tungsten compound.

To be able to precisely arrange or adjust the slat, the slat typically comprises a holding region with which the slat can be coupled to an adjusting facility. The adjusting facility is embodied to arrange or adjust the slat, and thus in particular the collimation region, precisely in the radiation field.

Owing to its properties, tungsten is difficult to join to other materials. In particular, tungsten has, for example compared to steel or copper, a low coefficient of thermal expansion or thermal coefficient. To prevent an input of heat resulting in stresses in the slat, the slat is typically manufactured from a single material, the material of the collimation region, in particular tungsten or a tungsten compound.

The collimation region, as well as the holding region of the slat, is thus typically made from the same material, in particular from tungsten or a tungsten compound. It is not necessary, however, to also manufacture the holding region from tungsten or a tungsten compound since the holding region is not arranged in the beam path and does not have to be embodied for attenuating the therapy radiation. Since tungsten is a very expensive material, it is of much interest to manufacture only the collimation region from tungsten or a tungsten compound.

US 2017/0148536 A1 describes a slat, wherein the holding region of the slat comprises a frame around the collimation region in which a tungsten plate is bordered. For this, firstly the individual parts, the holding region, including frame and the tungsten plate, have to be manufactured individually and subsequently joined.

### SUMMARY

This production process is very complex and thus time-consuming and typically also cost-intensive.

It is therefore the object of the present invention to provide a slat whose holding region is manufactured from a different material to the collimation region, it being possible to adhere to the above-mentioned precision requirements.

The object is achieved by a slat for collimating therapy radiation, by a collimator and by a method for producing a slat for collimating therapy radiation as claimed in the independent claims. Advantageous developments are stated in the dependent claims and in the following description.

The inventive achievement of the object will be described below both in relation to the claimed apparatuses and in relation to the claimed method. Features, advantages or alternative embodiments mentioned in this connection are likewise to be transferred to the other claimed subject matter, and vice versa. In other words, the physical claims (which are directed, for example, to an apparatus) can also be developed with the features, which are described or claimed in connection with a method. The corresponding functional features of the method are developed by corresponding physical modules.

### BRIEF DESCRIPTION OF THE DRAWINGS

The above-described properties, features and advantages of example embodiments of the present invention will become clearer and more comprehensible in conjunction with the following figures and their descriptions. The figures and descriptions are not intended to limit the invention and its embodiments in any way.

Identical components are provided with corresponding reference numerals in different figures. As a rule, the figures are not to scale.

In the drawings:

FIG. 1 shows a first exemplary embodiment of a slat for collimating therapy radiation,

FIG. 2 shows a second exemplary embodiment of a slat for collimating therapy radiation,

FIG. 3 shows an exemplary embodiment of a collimator,

FIG. 4 shows a first exemplary embodiment of a method for producing a slat for collimating therapy radiation,

FIG. 5 shows a second exemplary embodiment of a method for producing a slat for collimating therapy radiation, and

FIG. 6 shows a second exemplary embodiment of a method for producing a slat for collimating therapy radiation.

### DETAILED DESCRIPTION

One or more example embodiments of the present invention relates to a slat for collimating therapy radiation. The slat comprises a collimation region made from a first material and a holding region made from a second material. The collimation region and the holding region are connected together by a connection point. The first material is embodied for collimating therapy radiation. The holding region can be coupled to an adjusting facility for adjusting the slat.

In a particularly preferred embodiment of the invention, the therapy radiation is X-ray radiation. X-ray radiation describes electromagnetic radiation having an energy of more than 100 eV. X-ray radiation can be collimated, in particular, for radiation therapy. In radiation therapy a treatment area of an examination object is irradiated with ultra-hard or ultra-high-energy X-ray radiation (>1 MeV). In particular, the treatment area can be irradiated with X-ray radiation having an energy greater than or equal to 6 MeV.

In an alternative embodiment, the therapy radiation for the radiation therapy can be particle radiation, in particular proton radiation or heavy ion radiation or alpha radiation, etc.

In radiation therapy, for example tumors or heel spurs, tennis elbow, shoulder pain, osteoarthritis of the various joints vertebral hemangioma, etc. can be treated by way of irradiation with the therapy radiation. For this, the examination object, in particular a patient, is positioned in a radiation field of the therapy radiation. The examination object can be, in particular, a human or an animal. The examination object is positioned in such a way that an area to be treated or a treatment area is arranged in the radiation field. The radiation field describes an area which can be irradiated with therapy radiation in a plane perpendicular to a direction of propagation of the therapy radiation. In particular, the radiation field describes an area on the examination object or in a plane of the examination object which can be irradiated. The radiation field is delimited by the propagation of the therapy radiation. The propagation of the therapy radiation is described by a beam path. A projection of the beam path on the plane of the examination object can describe the radiation field. The therapy radiation is emitted by a source. If the therapy radiation is X-ray radiation, the source is an X-ray source. The X-ray source can be, in particular, a linear accelerator.

The slat is embodied to collimate the therapy radiation. In particular, the therapy radiation can be collimated with more than one slat. For this, the slat is arranged between the examination object and the source. The radiation field is shaped by the slat by way of the collimation of the therapy radiation in such a way that tissue and/or organs adjoining the treatment area, and which is/are positioned inside the radiation field, are shielded from the therapy radiation by the slat. In other words, an irradiated area on the examination object can be shaped by arranging or positioning the slat in the beam path. In other words, the radiation field is limited by the at least one slat to the irradiated area. In particular, the radiation field is limited in such a way that the area actually irradiated matches the treatment area. This step is referred to as “collimation”.

With collimation of the therapy radiation by way of the slat, an intensity of the therapy radiation on penetrating the slat is attenuated in such a way that the intensity of the therapy radiation behind the slat is negligible. Standards are specified in IEC 60601-2-1 (2016) for electron accelerators in the range of 1 MeV to 50 MeV for X-ray radiation. In particular, section 201.10.1.2.103.2.1 a specifies that the intensity of the X-ray radiation behind a slat should be at most 2% of the input intensity.

“Behind” the slat refers in this connection to the perspective of the slat from the position of the source. The slat is arranged in such a way that the therapy radiation penetrates the slat at least in some of the collimation region. The collimation region of the slat is extended for this purpose in the beam direction or direction of propagation of the therapy radiation. In particular, the extension of the slat in the beam direction will hereinafter be referred to as the “height” of the slat. In particular, the collimation region in the beam direction can comprise an extension between 5 cm and 9 cm. In particular, the extension of the slat in the beam direction can be 5 cm, 5.5 cm, 6 cm, 6.5 cm, 7 cm, 7.5 cm, 8 cm, 8.5 cm or 9 cm. The collimation region of the slat is thus embodied to be arranged at least partially in the beam path of the therapy radiation.

Perpendicular to the height, and therewith perpendicular to the beam path, the slat can have an extension between 0.5 mm and 1 cm. In particular, the slat perpendicular to the height and perpendicular to the beam path can comprise an extension between 1 mm and 6 mm. This extension will hereinafter be referred to as the “thickness” of the slat. In particular, the slat can thus be between 1 mm and 6 mm thick. In embodiments of the invention, the slat can be between 1.9 mm and 5.1 mm thick.

The collimation region is manufactured from the first material and the holding region from the second material. The first material and the second material are different from each other.

The holding region and the collimation region are connected together by the connection point. In particular, the holding region and the collimation region are rigidly or fixedly connected together by the connection point. In other words, a contact face of the holding region is connected to a contact face of the collimation region at the connection point. The connection point is embodied, in particular, in such a way that a stable connection can be ensured between the first and the second material. In particular, the connection point is embodied in such a way that no internal stress or stress occurs inside the slat at the connection point of the first and the second materials or this stress is minimal. In particular, the connection point can be embodied to withstand a force effect of up to 30 N/mm\hat{ }2 on milling or milling out of the slat. In particular, the connection point can be embodied in such a way that it withstands a force effect of up to 50 N/mm\hat{ }2.

The holding region is embodied to be coupled to an adjusting facility. The collimation region in the beam path can be adjusted or arranged or positioned to limit the radiation field by adjusting the holding region with the adjusting facility.

The first and/or the second material(s) satisfy at least one of the following criteria: radiation resistance (in particular up to approx. 250 kGy), operation temperature at least between 15 and 50° C., hardness of at least 50 HV (in particular of at least 70 HV, in particular of at least 75 HV), machinability, high corrosion resistance. In particular, the first and/or the second material(s) can satisfy all of these criteria.

The inventors have found that the material costs of the slat can be minimized by using different materials for the holding region and the collimation region. In particular, the inventors have found that the demands on the second material with regard to the attenuation of the therapy radiation are lower than on the first material. In particular, the inventors have found that a less expensive material can thus be chosen as the second material for the holding region. The inventors have also found that the second material can be lighter than the first material. In this way, the weight of the slat can be reduced. This can, in particular, simplify the operability of the slat. The inventors have found that the connection point can be easily and inexpensively produced.

According to one or more example embodiments of the present invention, the collimation region and the holding region are glued together at the connection point. Gluing takes place, in particular, with an epoxy resin-based adhesive.

To produce the connection point the adhesive is applied, in particular, to the contact faces of the holding region and/or the collimation region. The connection point is embodied by bringing the contact faces of the holding region and the collimation region together or in contact during curing of the adhesive. The connection point is thus embodied as a glued joint.

In embodiments of the invention, at least one of the contact faces can have been pre-treated before gluing. In other words, the contact face of the holding region and/or the contact face of the collimation region can have been pre-treated. In this way, a more stable connection of the adhesive to the contact face(s) can be achieved.

The adhesive can be, in particular, an epoxy resin-based adhesive.

Alternatively, an adhesive based on a different basis can be used for producing the connection point.

In particular, the adhesive can be a one- or a two-component adhesive.

The inventors have found that the connection point can be embodied inexpensively and a simple method can be embodied by gluing the holding region to the collimation region. The inventors have found that an epoxy resin-based adhesive is particularly radiation-resistant. The inventors have found that an epoxy resin-based adhesive does not become brittle, or becomes only slightly brittle, even with high exposure to radiation over a relatively long period. The inventors have found that an epoxy resin-based adhesive tolerates radiation of more than 250 kGy over a life of 10 years without becoming brittle.

According to one or more example embodiments of the present invention, the collimation region and the holding region are welded together at the connection point. Welding takes place, in particular, by friction welding, electron beam welding or laser welding.

With welding, a material-fit connection is embodied between the holding region and the collimation region by introducing a high amount of energy. This material-fit connection forms the connection point. The connection point is thus embodied, in particular, as a welded joint. In particular, the introduced energy has to be sufficiently high to transfer the first and the second materials, at least at the connection point, into a molten phase.

With friction welding, the energy is introduced, in particular, mechanically.

With electron beam welding and with laser welding, the energy is introduced by a temperature increase in the holding region and/or the collimation region. In particular, the energy can be introduced at certain points in this case.

The inventors have found that a stable connection point can be embodied between the holding region and the collimation region by welding. The inventors have found that a relatively low temperature increase or a temperature increase only at certain points is necessary due to friction welding and/or the introduction of the energy at certain points in the case of electron beam welding and/or laser welding. Stresses between the holding region and the collimation region due to the temperature increase can thus be prevented or reduced. In other words, stresses between the holding region and the collimation region owing to the different coefficients of thermal expansion of the first and second materials can be prevented or reduced. The inventors have found that welding in order to produce the connection point is a simple and inexpensive possibility of stably connecting together the holding region and the collimation region.

According to one or more example embodiments of the present invention, the collimation region and the holding region are soldered together at the connection point. Soldering takes place, in particular, by soft-soldering.

In other words, the connection point is embodied by a solder joint. For this, a solder is introduced between the holding region and the collimation region.

With soft-soldering, the connection point is embodied, in particular, at an operating temperature of below 450° C. In particular, a solder used for soft-soldering can melt in a range between 150° C. and 250° C.

In particular, the contact face of the holding region and/or the collimation region can be pre-processed before soldering of the holding region and the collimation region in order to guarantee good adhesion of the solder. In particular, at least the contact face of the first and/or the second material(s) can be coated for this purpose. In particular, coating can take place by way of a chemical coating or electroplating or by way of flame spraying.

With chemical coating, in particular the contact face of the first material can be chemically coated with nickel. In embodiments, a further coating of gold or silver or copper can be applied to the chemical nickel coating.

With electroplating, in particular the contact face of the first material can be silver-plated or copper-plated.

With flame spraying, copper or a copper-aluminum alloy (for example CuAl8) or tin bronze (for example CuSn6) can be applied in particular to the contact face of the first material.

In embodiments of the invention, the solder can be combined with a flux agent to form the connection point. For example, a flux agent which is based on a resin or a boron compound or a fluoride can be used. In particular, a solder based on tin (Sn) or a bismuth (Bi) can be used for producing the connection point. In particular, a solder can be used to which silver (Ag) or copper (Cu) is added. In less preferred embodiments, a solder can be used to which lead (Pb) is added.

In particular, a soldering foil can be used. In other words, the solder can be embodied as a foil, which is introduced between the holding region and the collimation region for soldering. The foil can have a uniform thickness. The foil can comprise, in particular, at least one face, which corresponds to the contact faces.

The inventors have found that soldering can easily and inexpensively produce a stable connection between the holding region and the collimation region. The inventors have found that a temperature necessary for soft-soldering is sufficiently low to avoid or minimize stresses between the holding region and the collimation region due to soldering. The inventors have found that using a soldering foil can ensure a uniform thickness of the connection point or the solder joint.

According to one or more example embodiments of the present invention, the first and the second materials are paramagnetic.

In other words, the first and the second materials cannot be magnetized. In particular, the magnetic permeability of the first and the second materials is less than 1.05 μ0. Here μ0 describes the permeability in a vacuum. In other words, “paramagnetic” means that the first and the second materials have a permeability is less than 1.05 μ0.

The inventors have found that by using paramagnetic materials the slat can also be used in a magnetic resonance imaging acronym: MRI) system. In particular, radiation therapy with monitoring via MRI is enabled in this way.

According to one or more example embodiments of the present invention, the first material is tungsten or a compound comprising tungsten.

A compound comprising tungsten will hereinafter also be referred to as a tungsten compound. The tungsten compound advantageously comprises a tungsten content of at least 90%. In particular, the tungsten compound can comprise a tungsten content of at least 95%.

In particular, the tungsten compound can also comprise nickel. In particular, an iron-nickel compound can form the binder or the matrix. Alternatively, a cupro-nickel compound can form a “binder” or a “matrix” in the tungsten compound if the slat is to be paramagnetic.

The inventors have found that tungsten is capable of adequately attenuating, in particular collimating, the therapy radiation, in particular the X-ray radiation, in radiation therapy with expedient spatial extension, in particular expedient height, of the collimation region. The inventors have found that for collimating the therapy radiation, at least the first material has to be embodied in such a way that the therapy radiation is adequately attenuated on penetrating the first material.

According to a further aspect of the compound, the second material is steel or aluminum or a copper alloy.

In particular, the second material can be less expensive than the first material. In particular, the second material is embodied to be glued or welded or soldered to the first material.

In particular, the second material can be machinable. In particular, the second material is corrosion-resistant. In particular, the second material can have a hardness of at least 50 HV, in particular of at least 70 HV, in particular of at least 75 HV.

In particular, the steel can be a stainless steel.

In particular, the copper alloy can be brass or bronze. In less preferred embodiments, the copper alloy can be cupro-nickel.

In alternative embodiments, the second material can be copper if the demands on the hardness of the second material are not so high.

In particular, the second material is embodied to enter into a permanent connection with the first material via the connection point. If the connection point is a glued joint or a solder joint, the second material is embodied to bind with the adhesive or with the solder to form the connection point.

The inventors have found that the second material does not have to satisfy any specific demands in respect of absorption capacity of therapy radiation, in particular X-ray radiation. The inventors have found that by using a less expensive second material, the slat can be produced less expensively. The inventors have found that steel, aluminum and/or a copper alloy satisfy the mechanical demands on the second material for use as a holding region of a slat. The inventors have found that machining of the second material is easier than that of the first material since the second material can be, in particular, less hard than the first material. The inventors have found that a production process of the slat can thus be accelerated and simplified. The inventors have found that milling the holding region from the second material is simplified compared to a holding region, which is produced from the first material. The inventors have also found that by using one of said materials as the second material, the weight of the slat can be reduced compared to a slat, which is composed completely of tungsten or a tungsten compound. In this way, operability, in particular, can be improved.

According to one or more example embodiments of the present invention, the slat comprises a guide element. The guide element is embodied by the first and the second materials or solely by the first material. The guide element is embodied to guide the slat in the adjusting facility.

The guide element is arranged, in particular, on the side of the slat facing the radiation source. Alternatively or in addition, a further guide element can be arranged on the side of the slat remote from the radiation source. The guide element is embodied to guide or stabilize the slat during adjustment with the adjusting facility. In particular, the guide element prevents rotating or tilting of the slat relative to the beam direction. In particular, the slat is adjusted or moved along the guide element further into the radiation field or the beam path or further out of the radiation field or the beam path on adjustment of the collimation region. In other words, the guide element, together with the adjusting facility, specifies a path along which the slat can be moved.

In particular, the guide element can be embodied to be guided in a guide system of the adjusting facility. The guide system comprises a counterpiece to the guide element. The guide system can be fixed relative to the source of the therapy radiation.

In particular, the guide element can be embodied as a guide rail or as a guide strip.

In particular, the guide element extends at least partially over the collimation region and at least partially the holding region. In particular, the guide element is formed from the first and the second materials.

Alternatively, the guide element can be embodied on at least one side of the slat solely by the second material. For this, part of the second material of the holding region can extend along the collimation region.

In particular, the guide element can be embodied by milling.

The inventors have found that the slat can be guided or stabilized by the guide element on adjustment by the adjusting facility. The inventors have also found that the guide element can be milled in after production of the connection point. The inventors have found that the connection point embodied as described above is sufficiently stable to withstand a force effect during milling. The inventors have found that in this way, the guide element can be embodied across the connection point.

One or more example embodiments of the present invention also relates to a collimator. The collimator comprises a plurality of above-described slats and an adjusting facility. The slats are coupled to the adjusting facility by their holding regions. The adjusting facility is embodied for adjusting each slat of the plurality of slats perpendicular to a contact face of the holding region and the collimation region.

The plurality of slats comprises at least two slats, which are embodied according to one of the above-described aspects. The plurality of slats is arranged side by side in the collimator. In other words, the slats are arranged side face to side face.

Each of the slats is coupled to the adjusting facility via their holding region. The holding region can be, for example, screwed or riveted or welded, etc. to the adjusting facility.

In particular, each slat can be adjusted with the adjusting facility in a plane parallel to its side face. In particular, each slat can be adjusted with the adjusting facility perpendicular to the contact faces of the holding region and the collimation region or perpendicular to the solder joint.

A side face of the slat is defined by the height of the slat and by the first and the second materials. In other words, the side face extends over the holding region and the collimation region. A slat comprises two side faces. The two side faces of a slat have a spacing relative to each other, which matches the thickness of the slat.

According to one or more example embodiments of the present invention, the adjusting facility can comprise an above-described guide system. In particular, the guide system can be embodied to guide the slats along their at least one guide element. In particular, the guide system is embodied to prevent lateral tilting of the slats. In other words, the guide system stabilizes an orientation of the slats.

The inventors have found that a plurality of slats can be arranged in one collimator. The inventors have found that the radiation field can be limited to the treatment area by adjusting the slats with the adjusting facility. The inventors have found that the holding region does not have to be arranged in the beam path for this. The inventors have found that for this reason, the second material does not have to satisfy the demand in respect of attenuation of the therapy radiation. The inventors have found that the holding region forms only a mechanical coupling of the collimation region with the adjusting facility.

One or more example embodiments of the present invention also relates to a method for producing a slat embodied as described above. The method comprises a method step of connecting a first block made from the first material to a second block made from the second material to form a combination block.

The first and the second blocks are, in particular, cuboidal or sickle-shaped. When the first and the second blocks are connected the connection point is embodied between the two blocks. The connection point is thus embodied by an at least approximately rectangular contact face of the first block and an at least approximately rectangular contact face of the second block. The combination block comprises the first and second blocks connected via the connection point.

The first block and the second block comprise at least one thickness, which matches the thickness of the slat. In other words, the first and the second blocks are at least 0.5 mm to 10 mm thick. In particular, the first and the second blocks can comprise a thickness between 1 mm and 6 mm. The thickness of the blocks describes an extension parallel to the contact faces. The contact faces are thus extended at least 0.5 mm to 10 mm, in particular at least 1 mm to 5 mm, in one direction. In embodiments, the contact faces can be extended at least 1 mm to 6 mm in one direction.

In particular, the at least approximately rectangular contact faces can be extended between 20 mm and 80 mm in the direction perpendicular thereto.

In particular in the direction perpendicular to the contact face, first block can comprise an extension between 100 mm and 180 mm. In particular, the extension of the first block perpendicular to the contact face can comprise 110 mm to 150 mm.

In particular in the direction perpendicular to the contact face, second block can comprise an extension between 50 mm and 150 mm. In particular, the extension of the second block perpendicular to the contact face can comprise 50 mm to 130 mm.

In an alternative embodiment, the second block can have the shape of a T-piece. The T-piece has three “arms”, which are connected together. Two of the arms are arranged in extension to each other. The third arm is arranged perpendicular to the other two arms. In this case, on connection the first block can be inserted between two arms in one of the rectangular recesses of the T-piece of the second block. The contact face is formed by two approximately rectangular faces. The extensions of the first block can be embodied approximately as described above. The T-piece of the second block is embodied in such a way that two arms of the T-piece enclose the collimation region. The length of these arms is adapted to the extension of the collimation region. The third arm can have, in particular, a length between 50 mm and 150 mm.

The inventors have found that the connection point is connected or embodied before a precise shaping of the slat on the basis of two blocks. The inventors have found that the action of heat due to connecting would potentially deform an already precisely shaped slat in such a way that that the precision requirements would no longer be satisfied. The inventors have found that this problem can be solved by connecting the first and the second materials before the slat is shaped. The inventors have found that a deformation on connection of the blocks can still be retrospectively compensated on shaping or milling out of the slat.

According to one or more example embodiments of the present invention, connecting comprises a method step of pre-treatment of least the faces of the first and the second blocks, which are to be connected together via the connection point. Pre-treatment comprises, in particular, grinding and/or smoothing and/or cleaning and/or chemical activation. Connecting also comprises a method step of gluing the first block to the second block, in particular via an epoxy resin-based adhesive.

The faces at which the first and the second blocks are to be connected or which are to be connected together via the connection point are referred to as contact faces.

In the method step of pre-treating, the contact faces are pre-treated in such a way that the contact faces can connect effectively with the adhesive. For this, the contact faces are ground and/or smoothed and/or cleaned and/or chemically activated.

In particular, a maximum roughness of the contact faces of Ra 3.2 (to ISO 21920-2) can be achieved by grinding or smoothing.

In particular, the contact faces can be treated with spirit and/or an industrial cleaner in the case of chemical activation.

In the method step of gluing, the first block is glued to the second block. The first and the second blocks are glued together at the contact faces. In other words, the contact face of the first block is glued to the contact face of the second block. In other words, the pre-treated face of the first block is glued to the pre-treated face of the second block. The adhesive is applied to the first and/or second contact face(s) and the two contact faces joined. The connection point between the first and the second blocks is embodied in this way.

The adhesive is, in particular as described above, an epoxy resin-based adhesive. The adhesive can be a one- or a two-component adhesive. Alternatively, the adhesive can also be based on a basis different to epoxy resin.

The inventors have found that a stable connection can be easily and inexpensively embodied between the first and the second blocks by gluing. The inventors have found that no complex process is necessary for this. The inventors have found that an appropriate pre-treatment of the contact faces can ensure that the adhesive connects effectively to the contact faces and a stable connection can be embodied between the contact faces. The inventors have found that there is no or only a slight introduction of heat into the first and second blocks with gluing. The inventors have found that the occurrence of stresses between the different materials of the first and the second blocks after production of the connection point owing to different coefficients of thermal expansion can be prevented in this way.

According to one or more example embodiments of the present invention, connecting comprises a method step of welding the first block to the second block. Welding takes place, in particular, via friction welding or electron beam welding or laser welding.

In particular, the contact face of the first block is thus welded or connected to the contact face of the second block. The connection point between the first and the second blocks at the contact faces is thus embodied by welding.

In particular, a material-fit connection between the first and the second blocks can be embodied by welding.

In particular, welding can take place in a vacuum or under inert gas. In particular, the vacuum can be embodied by an absolute air pressure of 10-100 mbar. The inert gas can be, for example, argon.

With friction welding, energy is introduced mechanically to connect or weld the two contact faces or blocks. With laser welding and with electron beam welding, the energy for producing the connection is introduced via an increase in the temperature at the weld joints. In particular, the temperature can be increased at certain points on the contact faces.

In particular, laser welding can be preferred compared to friction welding and electron beam welding.

The inventors have found that a stable connection between the first and the second blocks or the first and the second materials can be produced easily and inexpensively by welding the two material at the contact faces. The inventors have found that stresses, which can occur owing to different coefficients of thermal expansion of the first and the second materials, can be reduced or avoided by the mechanical introduction of the energy for welding in the case of friction welding. The inventors have found that stresses due to different coefficients of thermal expansion in the case of laser welding and electron beam welding can be prevented by heating the blocks only at certain points at the locations to be welded.

According to one or more example embodiments of the present invention, connecting comprises a method step of coating at least one face of the first block, which is to be connected to the second block via the connection point. Coating comprises, in particular, applying chemical nickel or electroplating or flame spraying. Connecting also comprises a method step of soldering the first block to the second block. Soldering takes place, in particular, via soft-soldering.

The faces of the first and the blocks, which are to be connected together, are referred to, as described above, as contact faces.

In the method step of coating, at least the contact face of the first block is coated. In particular, both contact faces, i.e. the contact face of the first block and the contact face of the second block, can be coated. In particular, the respective contact face is coated in such a way that a solder, which is to be used for soldering the two blocks, can bind to the first or second material via the coating.

In particular, coating can comprise applying chemical nickel or electroplating or flame spraying. In particular, the contact face of the first block made from the first material can be coated in such a way. In particular, the first material can be tungsten or a compound comprising tungsten.

In particular, nickel can thus be chemically applied to the contact face of the first block. In addition, in embodiments, further coats, for example of gold, silver and/or copper, can be applied to the nickel.

Alternatively, with electroplating or galvanizing, the contact face of the first block can be silver-plated or copper-plated.

Alternatively, with flame spraying, the contact face of the first block can be coated with a coating of copper or a coating of a copper-aluminum alloy (for example CuAl8) or a tin-bronze coating (for example CUSn6).

In advantageous embodiments of the invention, the contact face of the second block can be pre-treated at least mechanically or chemically.

In the method step of soldering, the first block and the second block are soldered together via their contact faces. The connection point between the first and the second blocks is embodied in the process. In particular, soldering can take place by way of soft-soldering. The first and the second blocks are soldered or joined together at a temperature of less than 450° C. In particular, a solder is used, which has a melting range between 150° C. and 250° C.

In particular, the solder can be used in conjunction or in combination with a flux agent.

In particular, a solder can be used, which is based on tin or bismuth. In particular, the solder can also comprise a silver or copper additive. Alternatively, in less preferred embodiments the solder can comprise a lead additive.

In particular, the solder can be embodied in the form of a foil solder. The foil solder can have a uniform thickness. The foil solder is introduced for the purpose of soldering between the contact faces of the first and the second blocks. The foil solder can, in particular, cover a face, which corresponds to the face of the contact faces.

In particular, the flux agent can be based on a resin and/or a boron compound and/or a fluoride and/or a zinc basis and/or an ammonium chloride basis.

Specifically, for example the following combination can be used for soft-soldering with flux agents: Braze Tex, Soldaflux 7000-basis: zinc chloride & ammonium chloride, effective temperature 150-400° C.; soft solder: Braze Tec Soldamoll 220 ((Sn 96.5 Ag 3.5)), strip 70.0×0.1 mm, melting range: 221° C.-230° C.

In particular, soldering can take place by running through a well-defined temperature curve. In other words, the first and the second blocks can be heated and cooled down again following a well-adjusted temperature curve, with the first and the second blocks being soldered at their contact faces. In particular, the temperature curve can be run through in a furnace run.

The inventors have found that soldering provides a simple and inexpensive possibility for embodying a stable connection between the first and the second blocks or materials. The inventors have found that with soft-soldering, the introduced temperature can be kept as low as possible and stresses owing to different coefficients of thermal expansion of the first and the second materials can be avoided or reduced in this way. In addition, stresses due to running through a well-defined temperature curve in the case of soldering can be reduced or avoided. The inventors have found that a preparatory coating of at least one contact face permits a more stable connection of the corresponding contact face to the solder. The inventors have found that wetting of the contact faces with the solder can be improved by the additional use of a flux agent.

According to one or more example embodiments of the present invention, the method comprises a method step of milling out at least one side face of the slat from the combination block.

The side face is embodied as described above.

In particular, the thickness of the combination block with this production process is equal to or only slightly larger than the thickness of the slat. The thickness of the combination block is specified by the thickness of the first or second block. In particular, the thickness of the combination block can be equal to or 5% or 10% greater than the thickness of the slat.

In particular, the shape of the side faces can be embodied or shaped by milling. In particular, a variable thickness of the slat over the height of the slat can be generated by shaping the side faces.

The inventors have found that the connection point remains stably unchanged even with a force effect due to milling. In other words, the inventors have found that the connection point also withstands a force effect due to milling.

According to one or more example embodiments of the present invention, the method comprises a method step of milling out at least one guide element from the combination block and/or a method step of milling out a contour of the holding region from the combination block.

The guide strip is embodied as described above. The guide strip can be milled out before milling out at least one side face of the slat from the combination block. Alternatively, the guide strip can be milled out after milling out at least one side face of the slat from the combination block.

The contour of the holding region describes a characteristic of the edges of the holding region, which are not in contact with the collimation region. In particular, the contour describes a contour of the side face of the slat in the holding region.

In particular, the contour is embodied in such a way that the holding region can be coupled to the adjusting facility. In particular, the contour of the holding region can form a web with which the holding region can be coupled to the adjusting facility.

In particular, the contour can be embodied in such a way that the holding region is embodied to be as lightweight as possible. In particular, the holding region can comprise at least one recess in the area of the side face of the slat embodied by the holding region. In other words, the contour can comprise at least one recess.

In particular, the method step of milling out the contour of the holding region can be carried out before milling out the at least one side face of the slat from the combination block. Alternatively, the method step of milling out the contour of the holding region can take place after milling out the at least one side face of the slat from the combination block. Alternatively, the contour of the holding region can take place partially before and partially after milling out the at least one side face of the slat from the combination block.

The inventors have found that the guide element and/or the contour of the holding region can be embodied by milling the combination block. The inventors have found that the connection point as described above is not impaired in terms of its stability by milling out.

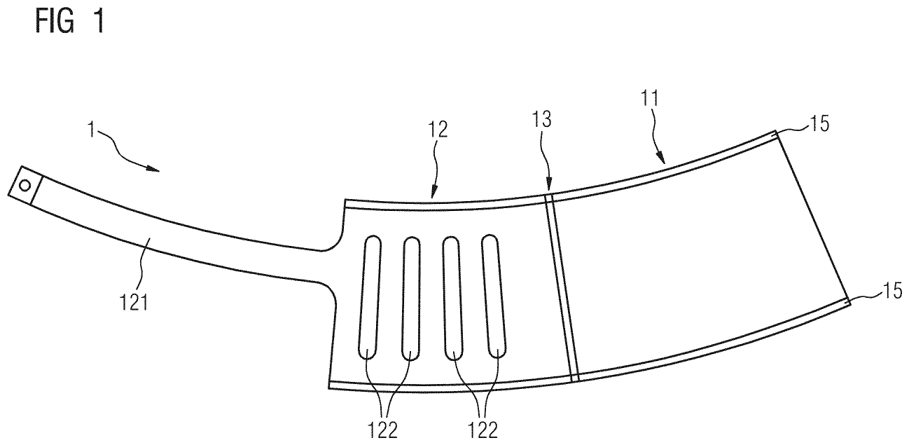

FIG. 1 shows a first exemplary embodiment of a slat 1 for collimating therapy radiation.

The slat 1 comprises a holding region 12 and a collimation region 11. The holding region 12 and the collimation region 11 are connected together via a connection point 13. In other words, the holding region 12 and the collimation region 11 are connected together. In particular, the holding region 12 and the collimation region 11 can be glued, welded or soldered together.

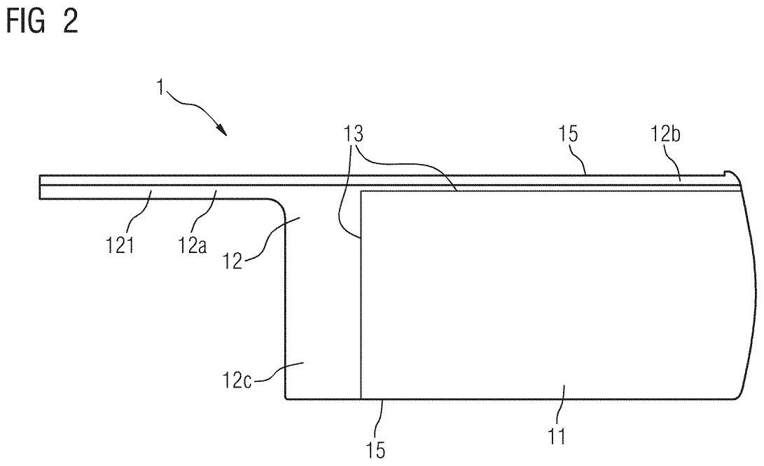

The slat 1 can be arranged in a beam path of therapy radiation for radiation therapy. In an advantageous embodiment of the invention, the therapy radiation can be X-ray radiation. In alternative embodiments, the therapy radiation can be particle radiation. The beam path describes a propagation of the therapy radiation. The beam path delimits a radiation field. The radiation field describes an area in a plane in which the therapy radiation propagates, or which the therapy radiation irradiates. In radiation therapy, a treatment area of an examination object is irradiated with the therapy radiation. In radiation therapy with X-ray radiation as the therapy radiation, typically ultra-hard X-ray radiation (>1 MeV) is used. In particular, X-ray radiation having an energy of greater than or equal to 6 MeV can be used. To ensure that only the treatment area is irradiated with the therapy radiation, the radiation field is limited by collimation of the therapy radiation using at least one slat 1. In particular, the slat 1 can be arranged in a collimator 2 as part of a plurality of slats 1, as represented in FIG. 2. In the represented orientation of the slat 1, a source of therapy radiation, in particular an X-ray source, is arranged above the slat 1. The examination object is arranged below the slat 1. The therapy radiation penetrates the slat 1 parallel to its height. In the represented orientation, the therapy radiation penetrates the slat 1 downwardly. FIG. 1 shows a view onto a side face of the slat 1. A thickness of the slat 1 describes an extension of the slat 1 into the image plane. The slat 1 can comprise a thickness between 0.5 mm and 10 mm. In particular, the slat 1 can comprise a thickness between 1 mm and 6 mm. In particular, the slat 1 can comprise a thickness between 1.9 mm and 5.1 mm. In particular, the thickness of the slat 1 can vary over the height. In particular, the slat 1 can be thinner at an upper edge of the side face than at a lower edge. In this case, “upper” and “lower” refer to the representation in FIG. 1. In other words, a cross-section perpendicular to the image plane through the slat 1 can be across-section of a truncated cone or a trapezoid. In particular, the thickness of the slat 1 is then defined by a maximum and a minimum thickness.

The collimation region 11 is manufactured from a first material. The first material is embodied for collimating the therapy radiation, in particular X-ray radiation. In other words, the first material is embodied to attenuate an intensity of the therapy radiation in such a way that the intensity of the therapy radiation after penetrating the collimation region 11 is negligible. In particular, if the therapy radiation is X-ray radiation, the intensity of the X-ray radiation through the penetration of the slat 1 can be attenuated to a maximum of 2% of the penetrating intensity.

In embodiments of the invention, the first material can be, in particular, tungsten or a compound comprising tungsten or a tungsten compound. The compound comprising tungsten comprises a tungsten content of at least 90%. The compound comprising tungsten comprises, in particular, a tungsten content of at least 95%. The compound comprising tungsten also comprises a binder or a matrix. The binder can be, in particular, iron-nickel or cupro-nickel.

The holding region 12 is embodied to be capable of being coupled to an adjusting facility. In particular, the holding region 12 can be coupled to the adjusting facility via a web 121. The web 121 can be embodied at any height of the slat 1. In particular, in the case of different slats 1 in a collimator 2 in FIG. 2, the web 121 can be embodied at different heights to enable easy or simple adjustment. In particular, in this way it is possible to prevent the slats 1 in the collimator 2 from hindering each other on adjustment with the adjusting facility. The holding region 12 can comprise at least one recess 122. The weight of the holding region 12 can be reduced by way of the recess 122. In particular, the weight can be reduced without impairing the stability of the holding region 12. In particular, a contour of the holding region 12 can be defined by the web 121 and/or the at least one recess 122. The holding region 12 is manufactured from a second material. In embodiments of the invention, the second material can comprise, in particular, steel or aluminum or a copper alloy.

The steel can be, in particular, stainless steel.

The copper alloy can be, for example, brass or bronze. In less preferred embodiments, the copper alloy can be cupro-nickel.

In optional embodiments of the invention, the first and the second materials can be paramagnetic. In particular, the magnetic permeability of the first and the second material is then less than 1.05 μ0. In particular, the binder of the collimation region 11 can then be cupro-nickel.

In embodiments of the invention, the first and/or the second material(s) can satisfy at least one of the following criteria: radiation resistance (in particular up to ca. 250 kGy), operation temperature at least between 15 and 50° C., hardness of at least 50 HV (in particular of at least 70 HV or 75 HV), machinability, high corrosion resistance. In particular, the first and/or the second material(s) can satisfy all of these criteria.

The collimation region 11 and the holding region 12 or the first and the second materials are connected together. In particular, a contact face of the collimation region 11 is connected to a contact face of the holding region 12. In particular, the collimation region 11 and the holding region 12 are connected together at the connection point 13.

In embodiments of the invention, the collimation region 11 and the holding region 12 are glued together at the connection point 13. In particular, the connection point 13 can thus be embodied by a glued joint. In particular, the collimation region 11 and the holding region 12 can be glued or connected at the connection point 13 with an epoxy resin-based adhesive. The adhesive can be a one- or a two-component adhesive. Alternatively, the adhesive can be based on a different basis to epoxy resin.

If the connection point 13 is embodied by gluing the collimation region 11 and the holding region 12, at least one of the contact faces, in particular at least the contact face of the collimation region 11, can have been pre-treated before gluing. For this, the contact face can be pre-treated by smoothing and/or grinding and/or cleaning and/or chemical activation.

In alternative embodiments of the invention, the collimation region 11 and the holding region 12 are welded together at the connection point 13. In other words, the connection point 13 can be embodied by a welded joint. Welding can comprise, in particular, friction welding, electron beam welding and/or laser welding.

In alternative embodiments of the invention, the collimation region 11 and the holding region 12 are soldered together at the connection point 13. In other words, the connection point 13 can be embodied by a solder joint. The solder joint is produced, in particular, by soft-soldering. In other words, the collimation region 11 and the holding region 12 are soldered via soft-soldering. In particular, soldering then takes place at a temperature of less than 450° C. In particular, a solder with a melting range between 150° C. and 250° C. can be used. In particular, a solder based on tin or bismuth can be used. In particular, silver or copper can be added to such a solder. In less preferred embodiments, lead can alternatively or additionally be added to such a solder. In particular, the solder can be a foil solder.

The solder can be combined with a flux agent. In particular, a flux agent based on a resin or a boron compound or a fluoride or a zinc basis or an ammonium chloride basis can be combined with the solder.

In particular, at least one of the contact faces can have been pre-treated, in particular coated, before soldering the holding region 11 to the collimation region 12. In particular, the contact face of the collimation region 12 can have been coated. In particular, the contact face can have been chemically coated or electroplated or have been coated by flame spraying. With chemical coating, the contact face of the collimation region 12 can have been coated with chemical nickel. In embodiments of the invention, a gold, silver or copper coating can be applied to the contact face in addition to the chemical nickel. With electroplating or galvanizing, the contact face of the collimation region 12 can be, in particular, silver-plated or copper-plated. With flame spraying, in particular a copper coating or a copper alloy coating (for example comprising CuAl8) or a tin-bronze coating (for example comprising CuSn6) can be applied to the contact face of the collimation region 12.

In embodiments of the invention, the slat 1 can comprise at least one guide element 15. The guide element 15 can be arranged at the upper edge or at the lower edge of the slat 1 or the side face of the slat 1. In particular, one guide element 15 can be arranged at the upper edge and one guide element 15 at the lower edge of the side face. The at least one guide element 15 can be a guide strip or a guide rail. The guide element 15 is embodied to prevent tilting of the slat 1 on adjustment of the slat 1 with the adjusting facility. In particular, the slat 1 can be adjusted or guided along the at least one guide element 15. The guide element 15 is embodied by the first and the second materials. In other words, the at least one guide element 15 extends at least partially over the holding region 12 and at least partially over the collimation region 11. In particular, the at least one guide element 15 can be milled into the first and second materials. In other words, the guide element 15 can be milled out of the first and second materials.

FIG. 2 shows a second exemplary embodiment of a slat 1 for collimating therapy radiation.

The second exemplary embodiment of the slat 1 differs solely in its shape, in particular in the shape of the holding region 12, from the first exemplary embodiment of the slat 1. The description relating to FIG. 1 thus applies analogously also to the second exemplary embodiment.

In contrast to the first exemplary embodiment in FIG. 1, the holding region 12 of the second exemplary embodiment is embodied by a T-piece. The T-piece comprises three “arms” 12a, 12b, 12c, which are connected together. One of the arms 12a forms the web 121. The other two arms 12b, 12c frame the collimation region 11 from two sides. The collimation region 11 is thus fitted or inserted in a rectangular recess of the T-piece of the holding region 12. The connection point 13 is embodied by two approximately rectangular faces. The contact face comprises the two faces. The extension of the collimation region 11 matches the extension of the collimation region 11 in the description relating to FIG. 1. In addition to the extension of the holding region 12 described in FIG. 1, the extension of the holding region 12 comprises the arm of the T-piece, which frames the collimation region 11 at the top in the drawing.

The guide element 15, which according to the drawing is arranged at the upper edge of the slat 1, is embodied by the second material. The guide element 15, which according to the drawing is arranged at the lower edge of the slat 1, is embodied partially by the first and partially by the second material.

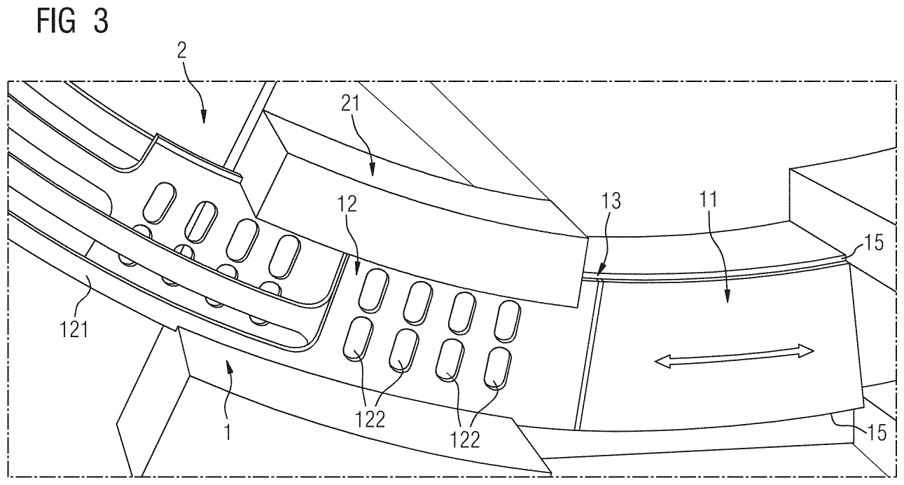

FIG. 3 shows an exemplary embodiment of a collimator 2.

The collimator 2 comprises a plurality of slats 1. In the represented exemplary embodiment, the collimator 2 comprises slats 1, which are embodied according to the first exemplary embodiment represented in FIG. 1. Alternatively, the collimator 1 can comprise slats 1, which are embodied according to the second exemplary embodiment in FIG. 2. Alternatively, the collimator 1 can also comprise other embodiments of the inventive slat 1. Each of the slats 1 is coupled to an adjusting facility by a web 121. The slats 1 can be adjusted according to the direction represented by the double arrow. The webs 121 of the different slats 1 are arranged on the corresponding slat 1 at different heights. In particular, adjustment of the slats 1 can be simplified in this way. In particular, it is possible to prevent the slats 1 from hindering each other on adjustment in this way.

In an alternative embodiment of the collimator 2, the webs 121 of all slats 1 can be arranged at the same height, in particular at an edge of the slat 1, as represented, for example, in FIG. 2. In this case, two slats 1 arranged adjacently in the collimator 2 are each arranged so as to be rotated by 180° about an axis parallel to a long side of the slat 1. In other words, in this case, two adjacent slats 1 are each arranged in the collimator 2 in such a way that in the case of one slat 1, the web 121 is arranged at the bottom and in the case of the other slat 1, the web 121 is arranged at top.

The collimator 2 also comprises a guide system 21. The slats 1 can be stabilized by the guide system 21 on adjustment. In particular, the slats 1 are guided in the guide system 21 along their guide elements 15. In particular, lateral tilting of the slats 1 can be prevented in this way.

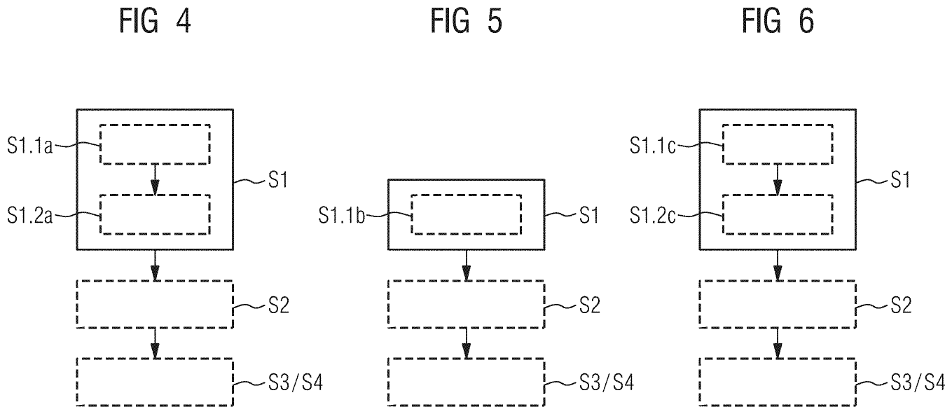

FIG. 4 shows a first exemplary embodiment of a method for producing a slat 1 for collimating therapy radiation.

In particular, a method for producing a slat 1 according to the first and second exemplary embodiments represented in FIGS. 1 and 2 is represented.

The method steps represented in broken lines are optional method steps, which can be encompassed by the method as a function of the properties of the produced slat 1.

The method comprises a method step of connecting S1 a first block made from the first material and a second block made from the second material to form a combination block.

On connecting S1, the connection point 13 is embodied between the first and the second blocks. The first and the second blocks are, in particular, cuboidal or sickle-shaped.

The first block and the second block comprise at least one thickness, which matches the thickness of the slat. In other words, the first and the second blocks are at least 0.5 mm to 10 mm, in particular at least 1 mm to 6 mm, thick. The thickness of the blocks describes an extension parallel to the contact faces. The contact faces are thus extended at least 0.5 mm to 10 mm, in particular at least 1 mm to 6 mm, in one direction.

In particular, the at least approximately rectangular contact faces can be extended between 20 mm and 90 mm in the direction perpendicular thereto.

In particular, first block can comprise an extension between 100 mm and 180 mm in the direction perpendicular to the contact face. In particular, the extension of the first block perpendicular to the contact face can comprise 110 mm to 150 mm.

In particular, second block can comprise an extension between 50 mm and 150 mm in the direction perpendicular to the contact face. In particular, the extension of the second block perpendicular to the contact face can comprise 50 mm to 130 mm.

The first and the second block can be approximately cuboidal. In particular, the second block is cuboidal if a slat 1 is produced according to the first exemplary embodiment in FIG. 1.

If a slat 1 is produced according to the second exemplary embodiment in FIG. 2, the second block is t-shaped.

If the second block is t-shaped, said extension can describe the extension of the arm 12a, which forms the web 121. In addition, the second block is extended in such a way that the other two arms of the T-piece can frame the collimation region 11, i.e. the first block. The extension of the second block is thus adapted to the extension of the first block.

The method step of connecting S1 comprises an optional method step of pre-treatment S1.1a of at least the faces of the first and the second blocks, which are to be connected together via the connection point, and an optional method step of gluing S1.2a the first block to the second block. The faces to be connected are referred to as contact faces as described above.

In the method step of pre-treatment S1.1a, in particular at least one of the contact faces of one of the two blocks is thus pre-treated. Preferably, both contact faces can be pre-treated.

With pre-treatment S1.1a, the contact faces are pre-treated in respect of their smoothness and/or roughness and/or cleaning and/or chemical activation. In particular, the contact faces can thus be smoothed and/or ground and/or cleaned and/or chemically activated. The contact faces are advantageously treated in such a way that they connect particularly effectively to an adhesive used in the method step of gluing S1.2a.

In particular, a maximum roughness of the contact faces of Ra 3.2 (to ISO 21920-2) can be achieved by grinding or smoothing.

In particular, the contact faces can be treated with spirit and/or an industrial cleaner in the case of chemical activation.

In the method step of gluing S1.2a, the first and the second blocks are glued together. The (in embodiments, pre-treated) contact faces of the first and the second blocks are glued together. The connection point 13 is embodied on gluing S1.2a. The connection point 13 is embodied in the form of a glued joint. Advantageously, the first and the second blocks are glued with an adhesive, which tolerates a radiation dose of more than 150 kGy over 10 years. Advantageously, the adhesive does not become brittle, or only slightly brittle, even with exposure to radiation at said level over 10 years. In particular, the adhesive is an epoxy resin-based adhesive. The adhesive can be a one- or two-component adhesive. Alternatively, the adhesive can also be based on a different basis.

The method can optionally comprise a method step of milling out S2 at least one side face of the slat 1 from the combination block.

In particular, in the method step of milling out S2, both side faces of the slat 1 can be milled out of the combination block. In other words, the side face of the slat 1 can be shaped by milling out S2 the side face. In particular, the wording “the side face is milled out of the combination block” is synonymous with the wording “the slat 1 is milled out of the combination block”. In particular, the thickness of the combination block matches the (maximum) thickness of the slat 1. Alternatively, the combination block is only slightly thicker, in particular a maximum of 10%, than the slat.

In an optional method step of milling out S3 at least one guide element 15, the at least one guide element 15 can be milled out of the combination block before milling out S2 the slat 1. In particular, the at least one guide element 15 is milled out parallel to the side face of the slat 1. The at least one guide element 15 is embodied as in the description relating to FIGS. 1 and 2.

Alternatively, the method step of milling out S3 the at least one guide element 15 can be performed after the optional method step of milling out S2 at least one side face of the slat 1.

The method also comprises an optional method step of milling out S4 a contour of the holding region 12 from the combination block.

In particular, the web 121 and the at least one recess 122 of the holding region 12 are milled out. In particular, the contour of the holding region 12 in the region of the combination block, which is embodied by the second material, is thus milled out. The method step of milling out S4 the contour of the holding region 12 can be performed before or after milling out S3 the at least one guide element 15.

In particular, the method step of milling out S4 the contour of the holding region 12 can be performed before milling out S2 the at least one side face of the slat 1. Alternatively, the contour of the holding region 12 can be milled out of the slat 1 that has already been cut or milled out. In other words, the method step of milling out S4 the contour of the holding region 12 can be performed after the method step of milling out S2 the at least one side face of the slat 1. Alternatively, the method step of milling out S4 the contour of the holding region 12 can be performed partially before and partially after the method step of milling out S2 the at least one side face of the slat 1. For example, the web 121 can be milled out beforehand and the at least one recess 122 thereafter.

FIG. 3 shows a second exemplary embodiment of a method for producing a slat 1 for collimating therapy radiation.

In principle, the method step of connecting S1 is embodied analogously to the description relating to FIG. 4. The optional method steps of milling out S2 at least one side face, of milling out S3 at least one guide element 15 and of milling out S4 a contour of the holding region 12 are embodied analogously to the description relating to FIG. 4. The basic description in respect of the first and the second blocks relating to FIG. 4 can be analogously transferred to the exemplary embodiment described below.

The method step of connecting S2 comprises an optional method step of welding S1.1b the first block to the second block. The first and the second blocks are welded together at their contact faces. The connection point 13 is embodied in the form of a welded joint.

Welding S1.1b can take place, in particular, by friction welding and/or electron beam welding and/or laser welding. Welding S1.1b can take place, in particular, under a vacuum or an inert gas atmosphere. In particular, the vacuum can be embodied by an absolute air pressure of 10-100 mbar. The inert gas can be, for example, argon.

FIG. 6 shows a second exemplary embodiment of a method for producing a slat 1 for collimating therapy radiation.

In principle, the method step of connecting S1 is embodied analogously to the description relating to FIG. 4. The optional method steps of milling out S2 at least one side face, of milling out S3 at least one guide element 15 and of milling out S4 a contour of the holding region 12 are embodied analogously to the description relating to FIG. 4. The basic description in respect of the first and the second blocks relating to FIG. 4 can be analogously transferred to the exemplary embodiment described below.

The method step of connecting S2 comprises an optional method step of coating S1.1c at least one face of the first block, which is to be connected to the second block via the connection point and an optional method step of soldering S1.2c the first block to the second block.

In the method step of coating S1.1c, thus at least the contact face of the first block is coated. Alternatively, the contact faces of the first and the second blocks can be coated. The corresponding contact face is coated in such a way that due to the coating, the first or second material binds better to a solder used in the method step of soldering S1.2c.

In particular, the contact face of the first block can be chemically coated and/or be electroplated and/or be coated via flame spraying.

With chemical coating or electroplating, a coating having a coating thickness between 0.005 mm and 0.1 mm can be applied to at least the contact face of the first block.

With flame spraying, a coating having a coating thickness for example between 0.1 mm and 0.5 mm can be applied to at least the contact face of the first block. Alternatively, higher coating thicknesses are possible with flame spraying.

With chemical coating, the contact face can be coated, in particular, with a chemical nickel. In embodiments of the invention, an additional coating of gold, silver and/or copper can be applied to the chemical nickel coating.

With electroplating or galvanizing, the contact face can be, in particular, silver-plated or copper-plated.

With flame spraying, in particular copper or a copper alloy (for example CuAl8) or tin-bronze (for example CuSn6) can be applied to the contact face.

In advantageous embodiments of the invention, the contact face of the second block can be at least mechanically and/or chemically pre-treated.

In the method step of soldering S1.2.c, the contact face of the first block is soldered to the contact face of the second block. The connection point 13 between the first and the second block is embodied in the form of a solder joint.

In particular, the first and the second blocks are soldered together via soft-soldering. In this case, soldering S1.2c takes place at an operating temperature of less than 450° C.

With soft-soldering, solders are used, which have a melting range between 150° C. and 250° C. In particular, a solder based on tin or bismuth can be used. Silver or copper can be added to the solder. In less preferred embodiments, lead can be added to the solder.

Flux agents which are based on a resin or a boron compound or a fluoride or a zinc basis or an ammonium chloride basis can be used as the flux agent.

Specifically, following combination of flux agent and solder can be used for soldering S1.2c the first and the second blocks: Braze Tex, Soldaflux 7000-Basis: zinc chloride & ammonium chloride, effective temperature 150-400° C.; soft solder: Braze Tec Soldamoll 220 ((Sn 96.5 Ag 3.5)), strip 70.0×0.1 mm, melting range: 221° C.-230° C.

For soldering, the first and the second block with the solder and optionally with the flux agent can be exposed by way of a furnace run to a well-defined temperature curve. The temperature curve is defined by a pre-heating time, a soldering time and a cooling time. During the pre-eating time the first and the second blocks as well as the solder and optionally the flux agent are brought to a required soldering temperature. During the soldering time the temperature is kept at or above the soldering temperature in order to carry the soldering process. During the cooling time the combination block connected by the solder and optionally by the flux agent is cooled to room temperature.

The solder can be introduced between the contact faces in particular in the form of a foil solder. The foil solder has a constant thickness. In particular, the foil solder comprises at least one face, which corresponds to the contact faces.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’, ‘interface’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

When a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Where it has not yet explicitly occurred but is expedient and within the meaning of the invention, individual exemplary embodiments, individual partial aspects or features thereof can be combined or replaced without departing from the scope of the present invention. Advantages of the invention described with reference to one exemplary embodiment also apply without being explicitly mentioned, and where transferable, to other exemplary embodiments.

1. A slat for collimating therapy radiation, comprising:
a collimation region made from a first material; and
a holding region made from a second material, wherein
the collimation region and the holding region are connected together by a connection point,
the first material is configured to collimate therapy radiation, and
the holding region is couplable to an adjusting facility for adjusting the slat.

2. The slat of claim 1, wherein
the collimation region and the holding region are glued together at the connection point.

3. The slat of claim 1, wherein
the collimation region and the holding region are welded together at the connection point.

4. The slat of claim 1, wherein
the collimation region and the holding region are soldered together at the connection point.

5. The slat of claim 1, wherein
the first material includes tungsten.

6. The slat of claim 1, wherein
the second material is steel, aluminum or a copper alloy.

7. The slat of claim 1, further comprising:
a guide element, wherein
the guide element includes the first material and the second material or only the second material, and
the guide element is configured to guide the slat in the adjusting facility.

8. A collimator, comprising:
a plurality of slats, each of the plurality of slats being the slat of claim 1; and
an adjusting facility, wherein
the slats are coupled to the adjusting facility by the holding regions of the slats, and
the adjusting facility is configured to adjust each slat of the plurality of slats perpendicularly to a contact face of the respective holding region and the collimation region.

9. A method for producing the slat of claim 1, the method comprising:
connecting a first block made from the first material and a second block made from the second material to form a combination block.

10. The method of claim 9, wherein the connecting comprises:
pre-treating at least faces of the first block and the second block, the faces of the first block and the second block are connectable via the connection point, wherein the pre-treating comprises at least one of grinding, smoothing, cleaning or chemical activation; and
gluing the first block to the second block.

11. The method of claim 9, wherein the connecting comprises:
welding the first block to the second block.

12. The method of claim 9, wherein the connecting comprises:
coating at least one face of the first block, the at least one face of the first block is connectable to the second block via the connection point, wherein the coating includes,
applying chemical nickel, electroplating or flame spraying; and
soldering the first block to the second block.

13. The method of claim 9, further comprising:
milling out at least one side face of the slat from the combination block.

14. The method of claim 9, further comprising at least one of:
milling out at least one guide element from the combination block; or
milling out a contour of the holding region from the combination block.

15. The slat of claim 4, wherein
the first material includes tungsten.

16. The slat of claim 15, wherein
the second material is steel, aluminum or a copper alloy.

17. The slat of claim 16, further comprising:
a guide element, wherein
the guide element includes the first material and the second material or only the second material, and
the guide element is configured to guide the slat in the adjusting facility.

18. The slat of claim 2, wherein the collimation region and the holding region are glued together with an epoxy resin-based adhesive.

19. The slat of claim 3, wherein the collimation region and the holding region are welded together by friction welding, electron beam welding or laser welding.

20. The slat of claim 4, wherein the collimation region and the holding region are soldered together by soft-soldering.
